(19) United States
(12) Patent Application Publication
(10) Pub. No.: US 2025/0148265 A1
(43) Pub. Date: May 8, 2025

(54) ARTIFICIAL INTELLIGENCE-BASED SUSTAINABLE MATERIAL DESIGN

(71) Applicant: Fujitsu Limited, Kawasaki-shi (JP)

(72) Inventor: Ramya MALUR SRINIVASAN, San Diego, CA (US)

(73) Assignee: Fujitsu Limited, Kawasaki-shi (JP)

(21) Appl. No.: 18/387,591

(22) Filed: Nov. 7, 2023

(51) Int. Cl. G06N 3/0455; G06N 3/0475; G06N 3/094

(52) U.S. Cl. CPC G06N 3/0455; G06N 3/0475; G06N 3/094

(57) ABSTRACT

In an embodiment, operations include receiving a dataset including information associated with scientific literature. The operations further include determining a set of materials and information associated the set of materials based on application of neural network models on the dataset. The operations further include generating embeddings for the set of materials indicative of features of each material and effect of each material on a living environment. The operations further include training a generative AI model based on the embeddings. The operations further include receiving a user input indicative of information associated with a queried material and generating embeddings for the queried material indicative of features of the queried material and its effect on the living environment. The operations further include determining sustainability information associated with the queried material based on application of the generative AI model on the embeddings generated for the queried material and rendering the sustainability information.

### FIELD

The embodiments discussed in the present disclosure are related to artificial intelligence (AI)-based sustainable material design.

### BACKGROUND

Advancements in the field of artificial intelligence (AI) have led to development of a multitude of AI-based frameworks and associated software that aid in sustainable material discovery, design, and synthesis. The AI-based frameworks have significantly expedited discovery, design, and synthesis of materials or combinations of materials, compared to legacy methods and techniques which would require years, if not decades, of effort for material discovery, design, and synthesis. The AI-based frameworks may be scalable, flexible, and self-contained, and enable usage of machine learning models (such as deep learning models). The machine learning models may be applied on scientific data with a specific focus on material information for discovery of sustainable or environmentally compatible materials. The associated software may provide solutions such as structure-based virtual screening of ultra-large chemical libraries for discovery of the sustainable materials and accelerating discovery of materials (such as polypeptide materials) for solving challenges such as artificial enzyme design, understanding of intrinsically disordered proteins, and so on. The AI-based frameworks also enable building element-wise graph neural networks that may be used for prediction of automatic synthesis of materials (such as synthesis of inorganic synthesis of recipes), retrosynthesis (synthesis of a generated material), learning of relationships between materials (entities) using knowledge graph, and so on.

However, generation of the scientific data and, subsequently, extraction of relevant information from the scientific data may be challenging for the deep learning models and graph neural networks. This may be because of challenges that may be involved in testing quality and resolution of the scientific data and determining relevance of the scientific data in aiding the discovery or synthesis of the materials. Moreover, the recognized or synthesized materials may not be environmentally compatible and may have an adverse impact on resources of a living environment.

The subject matter claimed in the present disclosure is not limited to embodiments that solve any disadvantages or that operate only in environments such as those described above. Rather, this background is only provided to illustrate one example technology area where some embodiments described in the present disclosure may be practiced.

### SUMMARY

According to an aspect of an embodiment, a method may include a set of operations, which may include receiving a dataset that includes information associated with scientific literature. The set of operations may further include applying one or more neural network models on the received dataset. The set of operations may further include determining a set of materials and information associated with each material of the set of materials, based on the application of the one or more neural network models. The set of operations may further include generating a first set of embeddings indicative of a first set of features of each material of the set of materials. The set of operations may further include generating a second set of embeddings associated with textual content that describes effects of the set of materials on resources of a living environment. The set of operations may further include training a generative artificial intelligence (AI) model based on the first set of embeddings and the second set of embeddings. The set of operations may further include receiving a user input indicative of information associated with a queried material. The set of operations may further include generating a third embedding indicative of a second set of features of the queried material and a fourth embedding associated with textual content that describes effects of the queried material on the resources of the living environment. The set of operations may further include applying the generative AI model on the third embedding and the fourth embedding. The set of operations may further include determining sustainability information associated with the queried material based on the application of the generative AI model. The set of operations may further include controlling a display device to render the sustainability information associated with the queried material.

The objects and advantages of the embodiments will be realized and achieved at least by the elements, features, and combinations particularly pointed out in the claims.

Both the foregoing general description and the following detailed description are given as examples and are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 is a diagram representing an example network environment related to artificial intelligence (AI)-based sustainable material design;

FIG. 2 is a block diagram that illustrates an exemplary electronic device for AI-based sustainable material design;

FIG. 3 is a diagram that illustrates an exemplary execution pipeline for AI-based sustainable material design;

FIG. 4 is a diagram that illustrates exemplary architecture that includes a language model and a generative AI model for AI-based sustainable material design; and

FIG. 5 is a diagram that illustrates a flowchart of an example method for AI-based sustainable material design;

all according to at least one embodiment described in the present disclosure.

### DESCRIPTION OF EMBODIMENTS

Some embodiments described in the present disclosure relate to methods and systems for artificial intelligence (AI)-based sustainable material design. Herein, the sustainable material design may involve reception of a dataset that includes information associated with scientific literature. Thereafter, one or more neural network models may be applied on the dataset. Based on the application of the one or more neural network models on the dataset, a set of materials and information associated with each material of the set of materials may be determined. Thereafter, a first set of embeddings, indicative of a first set of features of each material of the determined set of materials, may be generated. Further, a second set of embeddings, associated with textual content that describes effects of the determined set of materials on resources of a living environment, may be generated. Subsequently, a generative AI model may be trained based on the first set of embeddings and the second set of embeddings. After the training, a user input, indicative of information associated with a queried material, may be received. Upon reception of the user input, a third embedding and a fourth embedding may be generated. The third embedding may be indicative of a second set of features of the queried material. The fourth embedding may be associated with textual content that describes effects of the queried material on the resources of a living environment. Upon generation of the third embedding and the fourth embedding, the trained generative AI model may be applied on the third embedding and the fourth embedding. Thereafter, sustainability information associated with the queried material may be determined based on the application of the generative AI model on the third embedding and the fourth embedding. Finally, rendering of the sustainability information associated with the queried material may be on a display device may be controlled.

Traditional means of material design may generally require several years, if not decades, of effort for discovery, generation, or synthesis of sustainable materials. However, with emergence of big-data and advanced algorithms, AI-based frameworks have been developed for significantly expediting the discovery, generation, or synthesis of sustainable materials. For example, scalable, flexible, and self-contained frameworks may be used for application of deep learning models and methods on scientific data with a specific focus on material science for discovery of materials. The AI-based frameworks may work in tandem with software to enable structure-based virtual screening of ultra-large chemical libraries and accelerate discovery of new materials to address unknown challenges. For example, discovery of polypeptide materials may be accelerated to address challenges such as designing artificial enzymes, building understanding of intrinsically disordered proteins, and so on). For automating the synthesis of materials and building an understanding of retrosynthesis, graph neural networks are being used. For example, an element-wise graph neural network may be used to predict inorganic synthesis of recipes. The retrosynthesis (i.e., synthesis of a developed material) may be useful for understanding precursors of a particular reaction. Further, knowledge graphs may be used to facilitate learning of relations between different entities through graph neural networks. However, the AI-based frameworks may not include any mechanism to verify whether the discovered or synthesized materials will be environmentally compatible or have an adverse impact (such as extinction of existing resources due to increased carbon dioxide (CO₂) emissions of the materials) on the environment.

For discovery and synthesis of materials, natural language processing (NLP) techniques are being explored as NLP techniques facilitate extraction of information from datasets (such as, a chemical table). Based on the information entities constituting the materials may be recognized. For example, by use of the NLP techniques, a dataset may be generated. Based on the dataset, relationships between chemical interactions, that may be involved the synthesis of organic materials, may be identified. Thereafter, the organic materials may be synthesized by use of reactants involved in the chemical interactions. However, determination of quality and resolution of information included in the dataset and determination of relevance of the information for discovery or synthesis of sustainable or organic materials may be challenging.

According to one or more embodiments of the present disclosure, the technological field of environmentally compatible and sustainable material design may be improved by configuring a computing system (for example, an electronic device) in a manner that the computing system may generate environmentally compatible materials by use of a natural language model and a generative AI model. The computing system may receive a dataset that includes information associated with scientific literature. The scientific literature may be published in articles, journals, books, or presentations, associated with material science and chemical science. The scientific literature may be applied as input to neural network models for extraction of information that include entities (such as reactants or products) that may participate in chemical reactions and may be generated as outcomes of the chemical reactions. The extracted information may further include information associated with the entities such as organic structures of the entities, categories of the entities (catalysts, reactants, or products), time period for, and temperature at, which a chemical reaction involving the entities takes place, precursors associated with the chemical reactions involving the entities, and so on. Based on the information associated with the entities, the computing system may segregate the entities as sustainable (environmentally compatible) or unsustainable (environmentally incompatible or as potentially hazardous to the environment). The entities that are deemed unsustainable or potentially hazardous may be filtered and, consequently, entities that beneficial or sustainable may be determined.

The computing system may determine a set of materials based on the entities. Each material of the set of materials may include one or more entities. For each material of the set of materials, an embedding may be generated. Further, based on the information associated with each of the one or more entities constituting each material of the set of materials, another embedding may be generated. Thus, two embeddings may be generated for each material of the set of materials. The generation of the two embeddings may be based on application of a natural language model (e.g., a Bidirectional Encoder Representations from Transformers (BERT) model) on the set of materials and information associated with the set of materials. The generated embeddings associated with the set of materials may be used to train the generative AI model that may include a generator model and a discriminator model. Based on the embeddings, the discriminator model may be configured to classify whether each material of the set of materials is sustainable or hazardous. The generator model may be trained based on the embeddings and inputs from the discriminator model. The generator may be trained to generate an output for each material of the set of materials based on the embeddings associated with the corresponding material. The output may be such that reception of the output by the discriminator model may cause the discriminator model to classify the corresponding material as sustainable. On reception of a user input indicative of information associated with a queried material, the computing system may generate embeddings and feed the embeddings to the generative AI model. Based on the embeddings, the generated AI may predict whether the queried material is sustainable and a generate an explanation that outlines a rationale behind the prediction. Thus, the computing system of the current disclosure may predict, using a trained generative AI, whether a given material is sustainable and may also provide an explanation associated with a rationale behind the prediction. The prediction and the provided rationale may be useful for expedited design of sustainable and environmentally friendly new materials and compositions.

Embodiments of the present disclosure are explained with reference to the accompanying drawings.

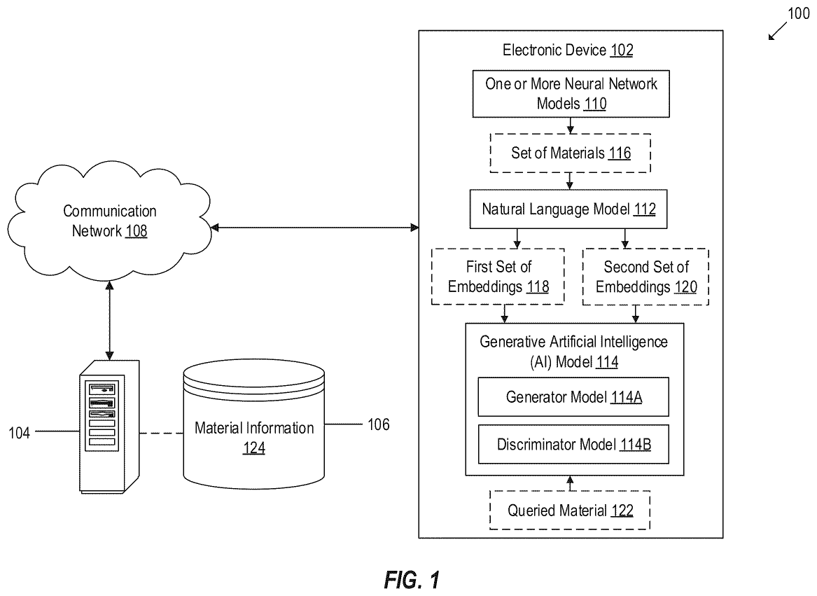

FIG. 1 is a diagram representing an example network environment related to artificial intelligence (AI)-based sustainable material design, according to at least one embodiment described in the present disclosure. With reference to FIG. 1, there is shown a network environment 100. The network environment 100 may include an electronic device 102 and a server 104 (that may host a database 106). The electronic device 102 and the server 104 may be communicatively coupled to each other, via a communication network (such as the communication network 108). The electronic device 102 may include one or more neural network models 110, a natural language model 112, and a generative AI model 114. The generative AI model 114 may include a generator model 114A and a discriminator model 114B. In FIG. 1, also shows that the electronic device 102 may include information such as, a set of materials 116, a first set of embeddings 118, a second set of embeddings 120, and a queried material 122. Further, the database 106 may include material information 124.

The electronic device 102 may include suitable logic, circuitry, interfaces, and/or code that may be configured to receive a dataset that may include information associated with a scientific literature. Further, the electronic device 102 may apply the one or more neural network models 110 on the dataset to determine the set of materials 116 and information associated with each material of the set of materials 116. Further, the electronic device 102 may generate the first set of embeddings 118 indicative of a first set of features of each material of the set of materials 116 and the second set of embeddings 120 associated with textual content that describes effects of the set of materials 116 on resources of a living environment. Thereafter, the electronic device 102 may train the generative AI model 114 based on the first set of embeddings 118 and the second set of embeddings 120. Further, the electronic device 102 may receive a user input that may be indicative of information associated with the queried material 122. The electronic device 102 may generate a third embedding that may be indicative of a second set of features of the queried material 122, and a fourth embedding that may be associated with textual content that describes effects of the queried material 122 on the resources of the living environment. The electronic device 102 may further apply the trained generative AI model 114 on the third embedding and the fourth embedding to determine sustainability information associated with the queried material 122 based on the application of the generative AI model 114, and control a display device to render sustainability information associated with the queried material 122. Examples of the electronic device 102 may include, but may not be limited to, a computing device, a smartphone, a mainframe machine, a server, a consumer electronic (CE) device, a computer workstation, and/or a device with a graph-processing capability (such as, a device with a set of graphic processor units (GPU)).

The server 104 may include suitable logic, circuitry, and interfaces, and/or code that may be configured to receive requests from the electronic device 102 for the dataset. The server 104 may be further configured to retrieve the dataset from the database 106 and transmit the dataset to the electronic device 102. In at least one embodiment, the server 104 may apply the one or more neural network models 110 (stored in the server 104) on the dataset (retrieved from the server 104) to determine the set of materials 116 and the information associated with each material of the set of materials 116. The server 104 may be further configured to generate the first set of embeddings 118 and the second set of embeddings 120 based on the set of materials 116 and the information associated with each material of the set of materials 116. Thereafter, the server 104 may transmit the first set of embeddings 118 and the second set of embeddings 120 to the electronic device 102. In some embodiments, the server 104 may be configured to receive the first set of embeddings 118, the second set of embeddings 120, and the queried material 122, from the electronic device 102. The server 104 may be further configured to train the generative AI model 114 (stored in the server 104) based on the generated first set of embeddings 118 and the generated second set of embeddings 120. Thereafter, the server 104 may determine sustainability information associated with the queried material 122 using the generative AI model 114 and transmit the determine sustainability information to the electronic device 102. The server 104 may be implemented as a cloud server and may execute operations through web applications, cloud applications, hypertext transport protocol (HTTP) requests, repository operations, file transfer, and the like. Other example implementations of the server 104 may include, but are not limited to, a database server, a file server, a web server, a media server, an application server, a mainframe server, a cloud computing server, and/or any device with a graph-processing capability (such as, a device with a set of graphic processor units (GPU)).

In at least one embodiment, the server 104 may be implemented as a plurality of distributed cloud-based resources by use of several technologies that may be well known to those ordinarily skilled in the art. A person with ordinary skill in the art will understand that the scope of the disclosure may not be limited to the implementation of the server 104 and the electronic device 102 as two separate entities. In certain embodiments, the functionalities of the server 104 can be incorporated in its entirety or at least partially in the electronic device 102, without a departure from the scope of the disclosure.

The database 106 may include suitable logic, circuitry, interfaces, and/or code that may be configured to store the material information 124. The material information 124 may correspond to the dataset that may include the information associated with the scientific literature. The database 106 may be derived from data off a relational or non-relational database, or a set of comma-separated values (csv) files in a conventional storage or a big-data storage. The database 106 may be stored or cached on a device, such as, the server 104 or the electronic device 102. The device storing the database 106 may be configured to receive a query for the material information 124. In response, the device storing the database 106 may be configured to retrieve and transmit the material information 124 to the electronic device 102. In accordance with an embodiment, the database 106 may be hosted on a plurality of servers stored at same or different locations. The operations of the database 106 may be executed using hardware including a processor, a microprocessor (for example, to perform or control performance of one or more operations), a field-programmable gate array (FPGA), or an application-specific integrated circuit (ASIC). In some other instances, the database 106 may be implemented using software.

The communication network 108 may include a communication medium via which the electronic device 102 and the server 104 may communicate with each other. The communication network 108 may be one of a wired connection or a wireless connection. Examples of the communication network 108 may include, but are not limited to, the Internet, a cloud network, a Cellular or Wireless Mobile Network (such as, Long-Term Evolution and 5G New Radio), a satellite network (such as, a network of a set of low-earth orbit satellites), a Wireless Fidelity (Wi-Fi) network, a Personal Area Network (PAN), a Local Area Network (LAN), or a Metropolitan Area Network (MAN). Various devices in the network environment 100 may be configured to connect to the communication network 100 in accordance with various wired and wireless communication protocols. Examples of the wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

In accordance with an embodiment, each of the one or more neural network models 110, the natural language model 112, the generative AI model 114 (including, for example, each of the generator model 114A and the discriminator model 114B) may be referred to as a neural network model. The neural network model may be a computational network or a system of artificial neurons that may be arranged in a plurality of layers. The neural network model may be defined by its hyper-parameters, for example, activation function(s), a number of weights, a cost function, a regularization function, an input size, a number of layers, and the like. Further, the layers may include an input layer, one or more hidden layers, and an output layer. Each layer of the plurality of layers may include one or more nodes (or artificial neurons). Outputs of all nodes in the input layer may be coupled to at least one node of hidden layer(s). Similarly, inputs of each hidden layer may be coupled to outputs of at least one node in other layers of the neural network. Outputs of each hidden layer may be coupled to inputs of at least one node in other layers of the neural network model. Node(s) in the final layer may receive inputs from at least one hidden layer to output a result. The number of layers and the number of nodes in each layer may be determined from the hyper-parameters of the neural network model. Such hyper-parameters may be set before or after training of the neural network model.

Each node may correspond to a mathematical function (e.g., a sigmoid function or a rectified linear unit) with parameters that are tunable during training of the neural network model. The set of parameters may include, for example, a weight parameter, a regularization parameter, and the like. Each node may use the mathematical function to compute an output based on one or more inputs from nodes in other layer(s) (e.g., previous layer(s)) of the neural network model. All or some of the nodes of the neural network model may correspond to the same or a different mathematical function. In training of the neural network model, one or more parameters of each node of the neural network model may be updated based on whether an output of the final layer for a given input (from the training dataset) matches a correct result in accordance with a loss function for the neural network model. The above process may be repeated for the same or a different input until a minimum of the loss function is achieved, and a training error is minimized. Several methods for training are known in art, for example, gradient descent, stochastic gradient descent, batch gradient descent, gradient boost, meta-heuristics, and the like.

In some embodiments, the neural network model may include electronic data, which may be implemented as a software component of an application executable on the electronic device 102. The neural network model may rely on libraries, external scripts, or logic/instructions for execution by a processing device included in the electronic device 102. In at least one embodiment, the neural network model may be implemented using hardware that may include a processor, a microprocessor (e.g., to perform or control performance of one or more operations), a FPGA, or an ASIC. Alternatively, in some embodiments, neural network model may be implemented using a combination of hardware and software. Examples of the neural network model may include, but are not limited to, a deep neural network (DNN), a convolutional neural network (CNN), an artificial neural network (ANN), a fully connected neural network, and/or a combination of such networks.

The one or more neural network models 110 may be applied on the received dataset that may include the information associated with the scientific literature (or the material information 124). In at least one embodiment, the received dataset (or the material information 124) may include articles and journals associated with technical domains, such as, material science domain and chemical science domain. The received dataset or the material information 124 may include one or more of entities, properties of the entities, and interactions that may be taking place between the entities. The one or more neural network models 110 may receive the dataset (or material information 124) as input. The one or more neural network models 110 may be trained to recognize named-entities and extract information associated with the recognized named entities from the dataset or the material information 124. The recognized named entities may include reactants, products, catalysts, and so on. The reactants may interact with each other for generation of products. The interactions between the reactants may be included as chemical reactions in the scientific literature. The chemical reactions may be triggered in certain environmental conditions (such as, temperature, pH level, or pressure) based on properties of the reactants. The chemical reactions may be facilitated by catalysts and involve one or more precursors. The information associated with the recognized named entities (extracted by the neural network) may include properties of each reactant and catalyst, properties of products that may be generated based on the chemical reactions involving the reactants, properties of the chemical reactions that involve the recognized named entities (i.e., the reactants and the catalysts), and/or conditions in which the chemical reactions may be triggered.

The one or more neural network models 110 may also be trained to filter the named entities based on whether the named entities are sustainable (environmentally friendly or harmless) or unsustainable (environmentally hazardous or harmful). In at least one embodiment, the filtering of the recognized named entities (i.e., reactants) may be based on outcomes (i.e., products) that may be generated due to interactions (i.e., chemical reactions) between the recognized named entities and the conditions in which the interactions may be triggered. For example, if a generated product is harmful or hazardous for the environment, then reactants that facilitate the generation of the product (due to an associated interaction) may be filtered. On the other hand, if reactants involved in a chemical reaction lead to generation of a sustainable product, then such reactants may be identified as useful or relevant.

The natural language model 112 may receive the set of materials 116 and information associated with the set of materials 116 as input. The set of materials 116 may include both sustainable and unsustainable materials. Each material of the set of materials 116 may include one or more reactants. Further, information associated with each material of the set of materials 116 may be obtained based on the information associated with the recognized named entities, i.e., the one or more reactants that constitute the corresponding material. The natural language model 112 may be trained to generate, as outputs, the first set of embeddings 118 and the second set of embeddings 120. The first set of embeddings 118 may include an embedding generated for each material of the set of materials 116. The embedding may indicate features of the corresponding material. The second set of embeddings 120 include an embedding that may be generated based on information associated with each material of the set of materials 116. The embedding may indicate an impact of the set of materials 116 on environment. Examples of the natural language model 112 may include, but are not limited to, a Bidirectional Encoder Representations from Transformers (BERT) model, a Generative Pre-trained Transformers (GPT) model, a Robustly Optimized BERT Pretraining Approach (ROBERTa) model, or a large language model (LLM).

The generative AI model 114 (i.e., each of the generator model 114A and the discriminator model 114B) may be trained based on the first set of embeddings 118, the second set of embeddings 120, and the information associated with the set of materials 116. The discriminator model 114B may be trained using embeddings associated with both sustainable materials and unsustainable materials included in the set of materials 116. The training may be such that the discriminator model 114B may classify whether an output, generated by the generator model 114A, is associated with a sustainable material. The generator model 114A may be trained to generate an output for the queried material 122 such that the discriminator model 114B may accurately predict whether the queried material 122 is sustainable. Thus, based on the training, the generative AI model 114 may be configured to predict whether the queried material 122 is sustainable. Examples of the generative AI model 114 may include, but are not limited to, a Generative Adversarial Network (GAN) model, a variational autoencoder (VAE) model, an auto-regressive model, a Generative Pre-trained Transformers (GPT) model, or a large language model (LLM).

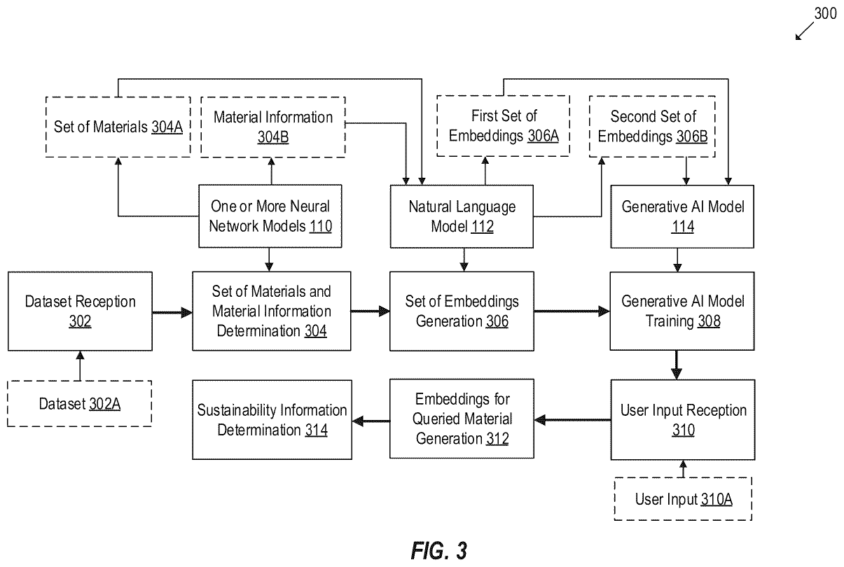

In operation, the electronic device 102 may be configured to receive a dataset that may include information associated with scientific literature. In accordance with an embodiment, the dataset may be received from the server 104 or the database 106 (via the server 104). In such embodiments, the dataset may correspond to the material information 124. The scientific literature may be associated with a technical domain, such as, material science domain or a chemical domain. The information associated with the scientific literature may include technical content. Details related to the reception of the dataset are described further, for example, in FIG. 3 (at 302).

The electronic device 102 may be further configured to apply the one or more neural network models 110 on the dataset. The one or more neural network models 110 may include a first neural network model and a second neural network model. Based on reception of the dataset (i.e., the information associated with the scientific literature) as input, the first neural network model may generate outputs. The outputs may include named entities such as reactants, products, catalysts, and so on, that may be involved in chemical reactions triggered in different circumstances. The outputs may further include information associated with each of the named entities. The named entities and the information associated with each of the named entities may be extracted from the information associated with the scientific literature.

The second neural network model may receive, as inputs, the extracted named entities and the extracted information associated with each of the named entities. The second neural network model may generate classification results that may indicate whether the named entities are sustainable or unsustainable. The classification may be based on the information associated with each of the named entities.

The electronic device 102 may be further configured to determine the set of materials 116 and information associated with each material of the set of materials 116. The determination may be based on the application of the one or more neural network models 110 on the dataset. Each material of the set of materials 116 may include one or more named entities extracted by the first neural network model. The information associated with each material of the set of materials 116 may be determined based on the information associated with the one or more named entities (extracted by the first neural network model) that may be included in the corresponding material.

In accordance with an embodiment, the determined set of materials 116 may include sustainable materials and unsustainable materials. The sustainable materials may include one or more sustainable named entities (classified as such by the second neural network model) while the unsustainable materials may include one or more unsustainable named entities (classified as such by the second neural network model). Details related to the determination of the set of materials and the material information are described further, for example, in FIG. 3 (at 304).

The electronic device 102 may be further configured to generate the first set of embeddings 118 that may be indicative of a first set of features of each material of the set of materials 116. In accordance with an embodiment, the generation of the first set of embeddings 118 may be based on an application of the natural language model 112 on the set of materials 116. Each embedding of the first set of embeddings 118 may be generated for each material the set of materials 116 and may indicate the first set of features for the corresponding material.

The electronic device 102 may be further configured to generate the second set of embeddings 120 that may be associated with textual content that describes effects of the set of materials 116 on the resources of a living environment. In accordance with an embodiment, the generation of the second set of embeddings 120 may be based on an application of the natural language model 112 on the information associated with each material of the set of materials 116. Each embedding of the second set of embeddings 120 may be generated for each material the set of materials 116 and may indicate the effects of the corresponding material on the resources of the living environment. Some of the embeddings of the second set of embeddings 120, which are generated for sustainable materials (that may include one or more sustainable named entities) included in the set of materials 116, may indicate a compatibility of the each of the sustainable materials with the environment. On the other hand, other embeddings of the second set of embeddings 120, which may be generated for unsustainable materials (that include one or more unsustainable named entities) included in the set of materials 116, may indicate an incompatibility of each of the unsustainable materials with the environment or a hazardous effect of each of the unsustainable materials on the environment. Details related to the generation of the embeddings (including, for example, the first set of embeddings and the second set of embeddings) are described further, for example, in FIG. 3 (at 306).

The electronic device 102 may be further configured to train the generative AI model 114 based on the first set of embeddings 118 and the second set of embeddings 120. The generative AI model 114, i.e., the generator model 114A and the discriminator model 114B, may be trained to predict whether a particular material is sustainable based on information associated with the material. The prediction may be based on the first set of embeddings 118 and the second set of embeddings 120. The generative AI model 114 further generate information that may indicate a rationale behind the prediction.

In accordance with an embodiment, the generator model 114A may be trained based on the first set of embeddings 118 and the second set of embeddings 120 to generate an output for the material. The output may be such that the discriminator model 114B classifies the material (for which the output was generated) as sustainable (i.e., environmentally friendly). Similarly, the discriminator model 114B may be trained based on the first set of embeddings 118 and the second set of embeddings 120 such the discriminator model 114B classifies the material as sustainable (i.e., environmentally friendly) or unsustainable (i.e., environmentally hazardous) accurately on reception of on the output (generated for the material by the generator model 114A). Details related to the training of the generative AI model are described further, for example, in FIG. 3 (at 308).

The electronic device 102 may be further configured to receive a user input that may be indicative of information associated with a queried material (such as, the queried material 122). The received user input may include an instruction to determine whether the queried material 122 is sustainable. The information, as indicated in the received user input, may be used to determine properties associated with the queried material 122. Based on the determined properties, one or more features of the queried material 122 may be determined, and an impact of the queried material 122 on the environment may also be determined. It may be noted that the queried material 122 may be a material of the set of materials 116. Details related to the reception of the user input are described further, for example, in FIG. 3 (at 310).

The electronic device 102 may be further configured to generate a third embedding indicative of a second set of features of the queried material 122 and a fourth embedding associated with textual content that describes effects of the queried material 122 on the resources of the living environment. The third embedding and the fourth embedding may be generated by use of the natural language model 112. In accordance with an embodiment, the queried material 122 may be fed as an input to the natural language model 112. Based on an application of the natural language model 112 on the queried material 122, the second set of features of the queried material 122 may be generated as an output of the natural language model 112 (indicated in the third embedding). Further, the application of the natural language model 112 on the information associated with the queried material 122 may generate the fourth embedding associated with the textual content that describes effects of the queried material 122 on the resources of the living environment. The third embedding and the fourth embedding may be used to determine sustainability of the queried material 122. Details related to the generation of embeddings (including, for example, the third embedding and the fourth embedding) for the queried material are described further, for example, in FIG. 3 (at 312).

The electronic device 102 may be further configured to apply the trained generative AI model 114 on the third embedding and the fourth embedding. The third embedding and the fourth embedding may be fed as inputs to the generative AI model 114. Additionally, the generator model 114A may receive an input noise and the received user input, i.e., the information associated with the queried material 122, as inputs. Based on the inputs, the generator model 114A may generate an output for the queried material 122. The discriminator model 114B may receive the output generated by the generator model 114A for the queried material 122, as an input (in addition to the generated third embedding and the generated fourth embedding).

The electronic device 102 may be further configured to determine (based on the application of the generative AI model 114) sustainability information associated with the queried material 122. The sustainability information may include at least one of a classification result obtained as an output of the discriminator model 114B and an explanation of the classification result. The output (i.e., the classification result) of the discriminator model 114B may correspond to a prediction of the generative AI model 114. The classification result may indicate whether the queried material 122 is classified as sustainable or unsustainable. The discriminator model 114B may classify the queried material 122 as sustainable if the queried material 122 is likely to be compatible with the environment or friendly to the environment. On the other hand, the discriminator model 114B may classify the queried material 122 as unsustainable if the queried material 122 is incompatible with, or likely hazardous to, the environment. Further, the explanation of the classification result may indicate a rationale behind the classification of the queried material 122 as sustainable or unsustainable. Details related to the determination of the sustainability information are described further, for example, in FIG. 3 (at 314).

The electronic device 102 may be further configured to control a display device to render the sustainability information associated with the queried material 122. In accordance with an embodiment, the electronic device 102 may transmit control instructions to the display device such that the display device, on reception of the control instructions from the electronic device 102, may render content that may be received from the electronic device 102. The display device may receive information that may include the sustainability information (for example, the output of the discriminator model 114B) and control instructions to render the sustainability information on a screen of the display device. On reception of the information, the received sustainability information associated with the queried material 122 may be rendered on a display screen of the display device.

Modifications, additions, or omissions may be made to FIG. 1 without departing from the scope of the disclosure. For example, the network environment 100 may include more or fewer elements than those illustrated and described in the present disclosure. In some embodiments, the functionality of each of the server 104 and the database 106, may be incorporated into the electronic device 102, without a deviation from the scope of the disclosure.

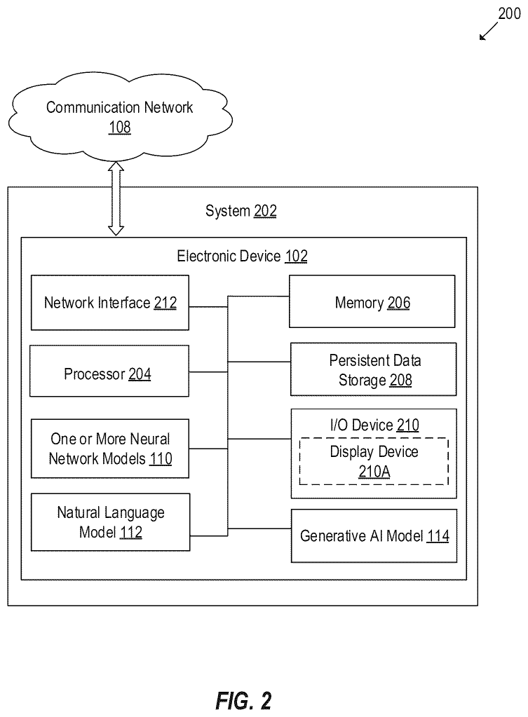

FIG. 2 is a block diagram that illustrates an exemplary electronic device for AI-based sustainable material design, in accordance with at least one embodiment described in the present disclosure. FIG. 2 is explained in conjunction with elements from FIG. 1. With reference to FIG. 2, there is shown a block diagram 200 of a system 202 that includes the electronic device 102. The electronic device 102 may include a processor 204, a memory 206, a persistent data storage 208, an input/output (I/O) device 210, and a network interface 212. In at least one embodiment, the memory 206 may store the one or more neural network models 110, the natural language model 112, and the generative AI model 114. In at least one embodiment, the I/O device 210 may include a display device 210A.

The processor 204 may include suitable logic, circuitry, and interfaces that may be configured to execute a set of instructions stored in the memory 206. The processor 204 may be configured to execute program instructions associated with different operations to be executed by the electronic device 102. The processor 204 may be configured to receive the dataset that may include information associated with the scientific literature. The processor 204 may be further configured to apply the one or more neural network models 110 on the dataset. The processor 204 may be further configured to determine the set of materials 116 and information associated with each material of the set of materials 116, based on the application of the one or more neural network models 110. The processor 204 may be further configured to generate the first set of embeddings 118 that may be indicative of a first set of features of each material of the set of materials 116. The processor 204 may be further configured to the second set of embeddings 120 that may be associated with textual content that describes effects of the set of materials 116 on resources of the living environment. The processor 204 may be further configured to train the generative AI model 114 based on the first set of embeddings 118 and the second set of embeddings 120. The processor 204 may be further configured to receive a user input indicative of information associated with the queried material 122. The processor 204 may be further configured to generate the third embedding that may be indicative of a second set of features of the queried material 122 and may be further configured to generate the fourth embedding that may be associated with textual content that describes effects of the queried material 122 on the resources of the living environment. The processor 204 may be further configured to apply the generative AI model 114 on the third embedding and the fourth embedding. The processor 204 may be further configured to determine sustainability information associated with the queried material 122 based on the application of the generative AI model 114. The processor 204 may be further configured to control the display device 210A to render the sustainability information associated with the queried material 122. The processor 204 may be implemented based on a number of processor technologies known in the art. Examples of the processor technologies may include, but are not limited to, a Central Processing Unit (CPU), X86-based processor, a Reduced Instruction Set Computing (RISC) processor, an Application-Specific Integrated Circuit (ASIC) processor, a Complex Instruction Set Computing (CISC) processor, a Graphical Processing Unit (GPU), a co-processor, or a combination thereof.

Although illustrated as a single processor in FIG. 2, the processor 204 may include any number of processors configured to, individually or collectively, perform or direct performance of any number of operations of the electronic device 102, as described in the present disclosure. Additionally, one or more of the processors may be present on one or more different electronic devices, such as different servers. In at least one embodiment, the processor 204 may be configured to interpret and/or execute program instructions, or process data that may be stored in the memory 206 or the persistent data storage 208. In some embodiments, the processor 204 be configured to may fetch program instructions from the persistent data storage 208 and load the program instructions in the memory 206. After the program instructions are loaded into the memory 206, the processor 204 may execute the program instructions.

The memory 206 may include suitable logic, circuitry, and interfaces that may be configured to store the one or more instructions to be executed by the processor 204. The one or more instructions stored in the memory 206 may be executed by the processor 204 to perform the different operations of the processor 204 (and the electronic device 102). The memory 206 that may store the dataset (e.g., the material information 124) that may include the information associated with the scientific literature, the set of materials 116 and the information associated with each material of the set of materials 116, the first set of embeddings 118, the second set of embeddings 120, the third embedding, the fourth embedding, and the sustainability information associated with the queried material 122. Examples of implementation of the memory 206 may include, but are not limited to, a CPU cache, a Hard Disk Drive (HDD), a Solid-State Drive (SSD), Random Access Memory (RAM), Read Only Memory (ROM), Electrically Erasable Programmable Read-Only Memory (EEPROM), and/or a Secure Digital (SD) card.

The persistent data storage 208 may include suitable logic, circuitry, and/or interfaces that may be configured to store program instructions executable by the processor 204. The persistent data storage 208 may include computer-readable storage media for carrying or having computer-executable instructions or data structures stored thereon. Such computer-readable storage media may include any available media that may be accessed by a general-purpose or special-purpose computer, such as the processor 204. By way of example, and not limitation, such computer-readable storage media may include tangible or non-transitory computer-readable storage media including Compact Disc Read-Only Memory (CD-ROM) or other optical disk storage, magnetic disk storage or other magnetic storage devices (e.g., Hard-Disk Drive (HDD)), flash memory devices (e.g., Solid State Drive (SSD), Secure Digital (SD) card, other solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 204 to perform a certain operation or group of operations associated with the electronic device 102.

The I/O device 210 may include suitable logic, circuitry, and interfaces that may be configured to receive inputs and render outputs based on the received inputs. For example, the I/O device 210 may receive an input that may trigger reception of the dataset that includes the information associated with the scientific literature. The I/O device 210 may further receive a user input indicative of information associated with the queried material 122. Further, the I/O device 210 may render outputs such as the determined set of materials 116, the information associated with each material of the set of materials 116, and the sustainability information that may be associated with the queried material 122. The I/O device 210 which may include various input and output devices, may be configured to communicate with the processor 204. Examples of the I/O device 210 may include, but are not limited to, a touch screen, a keyboard, a mouse, a joystick, the display device 210A, a microphone, and a speaker.

The display device 210A may include suitable logic, circuitry, and interfaces that may be configured to render the sustainability information associated with the queried material 122. The display device 210A may be a touch screen which may enable a user to provide user-inputs via the display device 210A. The touch screen may be at least one of a resistive touch screen, a capacitive touch screen, or a thermal touch screen. The display device 210A may be realized through several known technologies such as, but not limited to, a Liquid Crystal Display (LCD) display, a Light Emitting Diode (LED) display, a plasma display, or an Organic LED (OLED) display technology, or other display devices. In accordance with an embodiment, the display device 210A may refer to a display screen of a head mounted device (HMD), a smart-glass device, a see-through display, a projection-based display, an electro-chromic display, or a transparent display.

The network interface 212 may include suitable logic, circuitry, and interfaces that may be configured to facilitate communication between the processor 204 (i.e., the electronic device 102) and the server 104, via the communication network 108. The network interface 212 may be implemented by use of various known technologies to support wired or wireless communication of the electronic device 102 with the communication network 108. The network interface 212 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry. The network interface 212 may be configured to communicate via wireless communication with networks, such as the Internet, an Intranet, or a wireless network, such as a cellular telephone network, a wireless local area network (LAN), and a metropolitan area network (MAN). The wireless communication may be configured to use one or more of a plurality of communication standards, protocols and technologies, such as Enhanced Data GSM Environment (EDGE), wideband code division multiple access (W-CDMA), Long Term Evolution (LTE), 5th Generation (5G) New Radio (NR), Global System for Mobile Communications (GSM), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VOIP), light fidelity (Li-Fi), Worldwide Interoperability for Microwave Access (Wi-MAX), a protocol for email, instant messaging, and a Short Message Service (SMS).

Modifications, additions, or omissions may be made to the example electronic device 102 without departing from the scope of the present disclosure. For example, in some embodiments, the example electronic device 102 may include any number of other components that may not be explicitly illustrated or described for the sake of brevity.

FIG. 3 is a diagram that illustrates an exemplary execution pipeline for AI-based sustainable material design, in accordance with an embodiment of the disclosure. FIG. 3 is described in conjunction with elements from FIG. 1, and FIG. 2. With reference to FIG. 3, there is shown an execution pipeline 300. The exemplary execution pipeline 300 may include a sequence of operations that may be executed by the processor 204 of the electronic device 102 of FIG. 1 for discovery, design, and synthesis of sustainable materials, i.e., materials that are compatible or friendly with the environment. In the execution pipeline 300, there is shown a sequence of operations that may start from 302 and end at 314.

At 302, a dataset 302A, that includes information associated with scientific literature, may be received. In at least one embodiment, the processor 204 may be configured to receive the dataset 302A that may include the information associated with the scientific literature. The information associated with the scientific literature may be in a textual format or a multimedia format (such as, images, audios, or videos). The scientific literature may be associated with material science domain, biology domain, chemistry domain, bio-chemical domain, or environmental science domain. For example, the received dataset 302A may constitute information included in articles, study materials, books, presentations, and so on, that may be published by scientific or engineering community on subjects such as material science, chemicals, environmental science, or combinations of the above.

At 304, a set of materials 304A and material information 304B associated with the set of materials 304A may be determined based on the received dataset 302A. In at least one embodiment, the processor 204 may be configured to determine the set of materials 304A and the material information 304B associated with the set of materials 304A based on the received dataset 302A. The material information 304B may include information associated with each material of the set of materials 304A, determined from the received dataset 302A. The determination of the set of materials 304A and material information 304B may be based on application of a neural network-based analysis on the dataset 302A (i.e., the information associated with the scientific literature).

In accordance with an embodiment, the processor 204 may apply the one or more neural network models 110 on the received dataset 302A for the determination of the set of materials 304A and the material information 304B. For example, the processor 204 may apply a first neural network model of the one or more neural network models 110 on the received dataset 302A. The first neural network model may be a language model that may be configured to perform a named-entity recognition task on reception of the dataset 302A. The named-entity recognition task may include identification of named entities from the information associated with the scientific literature as included in the received dataset 302A. The named entities may be generated as outputs of the first neural network model. The identified named entities may include one or more of reactants, catalysts, and products. Thus, the processor 204 may identify a set of reactants based on the application of the first neural network model on the received dataset 302A. The processor 204 may further identify a set of products and a set of catalysts.

The named-entity recognition task may further include extraction of information associated with each of the named entities. The information associated with the named entities may include properties of the named entities, interactions between the named entities, and conditions in which the interactions between the named entities may be triggered. The interactions may include chemical reactions that may take place between reactants of the set of reactants. The occurrence of chemical reactions may be facilitated by catalysts of the set of catalysts and the chemical reactions may lead to generation of products of the set of products. The chemical reactions may be triggered in certain scenarios or conditions that may have certain impact on the environment. Therefore, the information associated with the named entities may include properties of each reactant of the set of reactants, properties of each product of the set of products, and properties of each catalyst of the set of catalysts. The information associated with the named entities may further include chemical reactions in which the set of reactants are involved, the set of products that are generated due to the chemical reactions, the set of catalysts that facilitate the chemical reactions, and conditions in which the chemical reactions are triggered.

Thus, the processor 204 may extract information associated with each identified reactant of the identified set of reactants based on the application of the first neural network model on the received dataset 302A. The information associated with each reactant of the identified set of reactants may include an organic structure of the corresponding reactant, a decay rate associated with the corresponding reactant, a biodegradability associated with the corresponding reactant, one or more catalysts that may facilitate a chemical reaction involving the corresponding reactant, one or more products generated due to the chemical reaction, a temperature requirement to trigger the chemical reaction, or one or more precursors that may be involved in the chemical reaction. The processor 204 may also extract information associated with each product of the set of products and information associated with each catalyst of the set of catalysts. The information associated with each product of the set of products may include organic structure decay rate, or biodegradability of the corresponding product.

In accordance with an embodiment, the processor 204 may apply a second neural network model on inputs that include the identified named entities and the extracted information associated with the identified named entities. The second neural network model may be a language-based classifier model that may be trained to perform a classification task. The classification task may involve classifying a named entity as sustainable or hazardous based on the information associated with the named entity. The named entity may be classified as sustainable if the named entity is environmentally friendly or compatible with the environment. On the other hand, the named entity may be classified as hazardous if the named entity is not compatible to the environment or is likely to be harmful for the environment. For determining whether a reactant of the identified set of reactants is sustainable, the processor 204 may apply the second neural network model of the one or more neural network models 110 on the identified set of reactants. Based on the application of the second neural network model, the processor 204 may select a subset of reactants from the identified set of reactants. The selection of the subset of reactants may be based on classification of each reactant of the subset of reactants as sustainable. The selected subset of reactants may be referred as a subset of sustainable reactants. Other reactants of the identified set of reactants (i.e., reactants not included in the selected subset of reactants) may be classified as hazardous. These reactants may constitute a subset of hazardous reactants.

For example, a reactant of the identified set of reactants may be classified as hazardous or unsustainable if the reactant is not biodegradable, has a low decay rate (or a high half-life), is involved in a chemical reaction leading to generation of a product that could be hazardous to the environment. On the other hand, a reactant of the identified set of reactants may be classified sustainable if the reactant is biodegradable or involved in a chemical reaction leading to generation of a sustainable or desirous product.

In accordance with an embodiment, the set of materials 304A may be determined based on the identification of the set of reactants and the selection of the subset of reactants. Each material of the set of materials 304A may include one or more reactants of the set of reactants. Further, the set of materials 304A may include a subset of sustainable materials and a subset of hazardous materials. Each sustainable material of the subset of sustainable materials may include one or more reactants of the selected subset of reactants (subset of sustainable reactants). However, each hazardous material of the subset of hazardous materials may include one or more reactants of the identified set of reactants that are classified as hazardous. In some embodiments, the hazardous materials may be filtered, and the subset of sustainable materials may be determined as relevant or useful.

In accordance with an embodiment, the material information 304B may include information associated with each material of the set of materials 304A. Thus, the material information 304B may include information associated with each material of the subset of sustainable materials and information associated with each material of the subset of hazardous materials. The information associated with each material of the set of materials 304A may be determined based on the extracted information associated with each reactant of the one or more reactants (of the identified set of reactants) that may be included in the corresponding material.

At 306, embeddings associated with the determined set of materials 304A may be generated. In at least one embodiment, the processor 204 may be configured to generate the embeddings associated with the determined set of materials 304A. The generated embeddings may include a first set of embeddings 306A and a second set of embeddings 306B. Each embedding of the first set of embeddings 306A and each embedding of the second set of embeddings 306B may be generated for each material of the set of materials 304A. The first set of embeddings 306A may be generated based on an application of the natural language model 112 on the determined set of materials 304A. The first set of embeddings 306A may be indicative of a set of features of each material of the determined set of materials 304A. The second set of embeddings 306B may be generated based on an application of the natural language model 112 on the material information 304B. The second set of embeddings 306B may be associated with textual content that describes effects of the determined set of materials 304A on resources of a living environment. Examples of the natural language model 112 may include, but are not limited to, a Bidirectional Encoder Representations from Transformers (BERT) model, a Generative Pre-trained Transformers (GPT) model, a Robustly Optimized BERT Pretraining Approach (ROBERTa) model, or a large language model (LLM).

In accordance with an embodiment, the processor 204 may apply the natural language model 112 on each material of the determined set of materials 304A. Based on the application, an embedding of the first set of embeddings 306A for the corresponding material may be generated. The generated embedding may indicate the features of the corresponding material. The features may indicate that the corresponding material is sustainable or hazardous based on classification of one or more reactants constituting the corresponding material as sustainable or hazardous. The processor 204 may further apply the natural language model 112 on the information associated with each material of the determined set of materials 304A. Based on the application, an embedding of the second set of embeddings 306B for the corresponding material may be generated. The generated embedding may indicate the impact of the corresponding material on the environment. The impact may indicate whether the corresponding material is useful (for example, if efficiency of products including the corresponding material improves) or the harmful for the environment (for example, if the corresponding material releases gases in certain conditions that are toxic or reduces efficiency of products that may include the corresponding material).

At 308, the generative AI model 114 may be trained based on the first set of embeddings 306A and the second set of embeddings 306B. In at least one embodiment, the processor 204 may be configured to train the generative AI model 114 based on the first set of embeddings 306A and the second set of embeddings 306B. Examples of the generative AI model 114 may include, but are not limited to, a Generative Adversarial Network (GAN) model, a variational autoencoder (VAE) model, an auto-regressive model, a Generative Pre-trained Transformers (GPT) model, or a large language model (LLM). The generative AI model 114, after training, may predict sustainability information associated with a material and generate an explanation that includes a rationale behind the prediction. In an embodiment, the generative AI model 114 may correspond to a conditional generative adversarial network (GAN) model that includes the generator model 114A and the discriminator model 114B. In an embodiment, the generator model 114A may be trained, while, at the same time, the discriminator model 114B may be idle (i.e., not getting trained). Further, the discriminator model 114B may be trained prior to or after the training of the generator model 114A, such that during the time interval of training of the discriminator model 114B, the generator model 114A may be idle (i.e., not getting trained). The generator model 114A may be trained to generate an output for each material of the set of materials 304A such that the discriminator model 114B classifies each material of the set of materials 304A as sustainable. On the other hand, the discriminator model 114B may be trained to accurately classify each material of the set of materials 304A as sustainable or hazardous.

In accordance with an embodiment, the processor 204 may train the generator model 114A based on the first set of embeddings 306A, the second set of embeddings 306B, and the information associated with each material of the set of materials 304A (i.e., the material information 304B). The generator model 114A may be trained further based on a random input and a generator loss. The random input may be random noise (for example, white noise) generated using a Gaussian noise model. For a material of the set of materials 304A, the generator model 114A may be applied on the random input, a first embedding of the first set of embeddings 306A generated for the material, and a second embedding of the second set of embeddings 306B generated for the material. Based on the application of the generator model 114A, an output may be generated for the material. The discriminator model 114B may be applied on the generated output for generation of a generator loss. The generator model 114A may be trained further based on the generator loss to generate an updated output for the material such that application of the discriminator model 114B on the updated output may result in minimization of the generator loss and classification of the material as sustainable. The generator model 114A may be similarly trained to generate outputs for each of the other materials of the set of materials 304A.

Similarly, the processor 204 may train the discriminator model 114B based on the first set of embeddings 306A, the second set of embeddings 306B, and the information associated with each material of the set of materials 304A (i.e., the material information 304B). The discriminator model 114B may be trained further based on the output that may be generated by the generator for each material of the set of materials 304A and a discriminator loss. On application of the discriminator model 114B on an output generated by the generator model 114A for a material (of the set of materials 304A), a first embedding (of the first set of embeddings 306A) generated for the material), and a second embedding (of the second set of embeddings 306B) generated for the material, the discriminator model 114B may generate a classification result as output. The classification result may be a value which indicates whether the material is sustainable or hazardous and an extent to which the material is sustainable or hazardous. Based on the classification result, the discriminator model 114B may generate a discriminator loss. The discriminator model may be trained further based on the discriminator loss such that the material is accurately classified as sustainable or hazardous. The discriminator model 114B may be similarly trained to accurately classify each of the other materials of the set of materials 304A as sustainable or hazardous.

At 310, a user input 310A, indicative of information associated with a queried material, may be received. In at least one embodiment, the processor 204 may be configured to receive the user input 310A indicative of the information associated with the queried material. The reception of the user input 310A may be based on a requirement to determine whether the queried material is sustainable or hazardous. The information associated with the queried material may be extracted from scientific literature using a language-based model (such as, the first neural network model). The queried material may include one or more reactants or a combination of reactants or products. In some embodiments, the queried material may be a material of the set of materials 304A.

At 312, embeddings associated with the queried material may be generated based on information associated with the queried material. In at least one embodiment, the processor 204 may be configured to generate embeddings associated with the queried material based on the information associated with the queried material. The generated embeddings may include a third embedding and a fourth embedding. The third embedding may indicate a set of features of the queried material and the fourth embedding may be associated with textual content that describes effects of the queried material on the resources of the living environment.

In accordance with an embodiment, the processor 204 may apply the natural language model 112 on the queried material for the generation of the third embedding. The processor 204 may further apply the natural language model 112 on the information associated with the queried material for the generation of the fourth embedding.

In some embodiments, the queried material may be a material of the set of materials 304A. In such embodiments, the generated third embedding may be an embedding of the first set of embeddings 306A and the generated fourth embedding may be an embedding of the second set of embeddings 306B.

At 314, sustainability information associated with the queried material may be determined based on the third embedding and the fourth embedding. In at least one embodiment, the processor 204 may be configured to determine the sustainability information associated with the queried material based on the third embedding and the fourth embedding. The sustainability information associated with the queried material may correspond to a first indication that may specify whether the queried material is sustainable or hazardous and a second indication that explains a rationale behind the first indication. The sustainability information may be determined based on application of the generative AI model 114 on the third embedding and the fourth embedding.

In accordance with an embodiment, the generator model 114A may receive a first set of inputs. The first set of inputs may include a random input, the generated third embedding (i.e., the set of features of the queried material), and the generated fourth embedding (i.e., the impact of the queried material on the environment). Based on the first set of inputs, the generator model 114A may generate an output. Thereafter, the discriminator model 114B may receive a second set of inputs. The second set of inputs may include the output generated by the generator model 114A, the third embedding, and the fourth embedding. Based on the second set of inputs, the discriminator model 114B may generate a classification result that indicates whether the queried material as sustainable or hazardous. The generated classification result may be prediction of the generative AI model 114 that corresponds to the first indication. The discriminator model 114B may further generate a rationale behind the first indication (i.e., the generated classification or prediction).

For example, the queried material may be material that may be generated based on oxidation of oil molecules with oxygen molecules. The information associated with the material may indicate that the material may release hazardous chemicals in water bodies if the material is used in an equipment that is deployed in water bodies and comes in contact with water. Based on an application of the natural language model 112 on the queried material and the information associated with the material, embeddings may be generated for the material and the information associated with the material. Based on an application of the generative AI model 114 on the generated embeddings, the discriminator model 114B may classify the material as hazardous and generate a rationale behind the classification that may be “Material comes with the risk of hazardous chemical release into water bodies”.

In another example, the queried material may be a synthesized fertilizer and the information associated with the queried material may indicate that the queried material releases excessive ammonia. The queried material may be classified as hazardous and a rationale that is generated may be “synthesized fertilizer comes with the risk of hazardous chemical release into atmosphere”. In another example, the queried material may be a synthesized drug and the information associated with the queried material may indicate that the queried material releases dioxins. The queried material may be classified as hazardous and a rationale that is generated may be “synthesized fertilizer comes with the risk of side-effects in patients”.

In another example, the queried material may be a material that can be used for assessing industrial wastes and the information associated with the queried material may indicate that the queried material is generated from plant-based gums. The queried material may be classified as sustainable and a rationale that is generated may be “material is sourced from plant-based materials”. In another example, the queried material may be a synthesized fertilizer and the information associated with the queried material may indicate that the queried material is produced from natural wastes. The queried material may be classified as sustainable and a rationale that is generated may be “synthesized fertilizer is produced from natural wastes”.

In another example, the queried material may be a synthesized drug and the information associated with the queried material may indicate that the queried material is generated from plant oils. The queried material may be classified as sustainable and a rationale that is generated may be “synthesized fertilizer is sourced from plant herbs and does not cause side-effects”.

Embodiments of the disclosure may facilitate generation of materials while taking into account an impact of the generated materials on the resources of the living environment. For the generation of sustainable materials that are compatible and environmentally friendly in nature, the embodiments may leverage natural language models and generative AI-models. The usage of AI-based models allows an accurate classification of materials in terms of sustainability of the materials with the environment, thereby protecting the environment from potentially hazardous effects that unsustainable materials may have on the environment. The AI-based models further facilitate in providing explanations behind predictions indicative of sustainability of materials. The explanations include rationales behind each of the predictions in terms of the impact the materials may have on the environment. The generation of the rationales behind the predictions may allow creation of a knowledge base associated with properties of the materials and usage of the knowledge base to create training data for training the AI-based models such that accuracy of future predictions by the AI-based models is enhanced.

The embodiments allow designing of new materials in an ethical manner that minimizes or nullifies hazardous effects, if any, of the materials on the environment or climate. The embodiments provide a conditional GAN model that may be trained based on embeddings indicative of features of the materials and effects of the materials on the resources of the living environment. The training allows filtering of materials that are unwanted, incompatible, or hazardous to the environment, and facilitates generation of new materials that may be environmentally friendly. The usage of the embeddings may facilitate incorporation, by the conditional GAN model, of time-varying environmental constraints by encoding dynamic reactions between chemicals (i.e., reactants) to aid in sustainable material discovery, design, and synthesis. The generation of the materials may be aligned with the United Nations (UN) Sustainability Development Goals (SDGs).

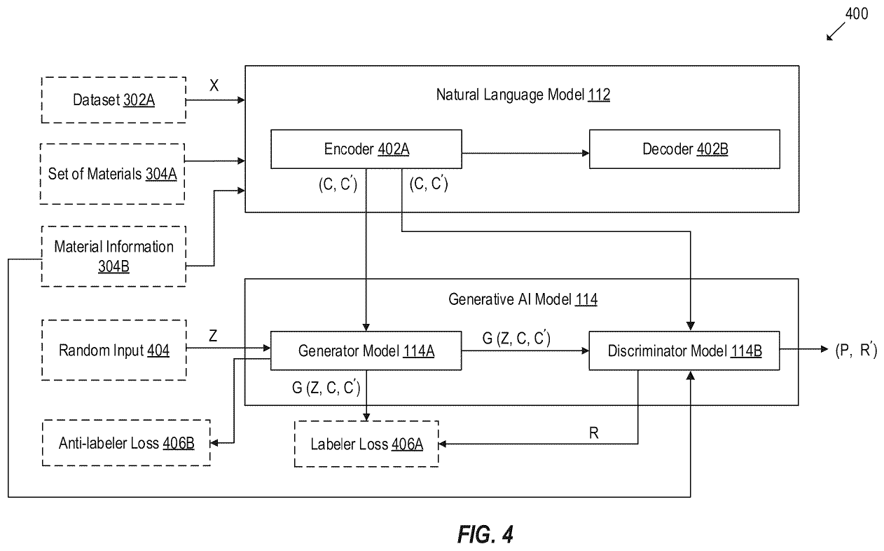

FIG. 4 is a diagram that illustrates exemplary architecture that includes a language model and a generative AI model for AI-based sustainable material design, in accordance with an embodiment of the disclosure. FIG. 4 is described in conjunction with elements from FIG. 1, FIG. 2, and FIG. 3. With reference to FIG. 4, there is shown an exemplary architecture 400. The exemplary architecture 400 may include the natural language model 112 and the generative AI model 114 of FIG. 1. The natural language model 112 may receive the dataset 302A (represented as “X”), the set of materials 304A, and the material information 304B as inputs. As discussed in FIG. 3, the set of materials 304A and the material information 304B may be determined based on the application of the one or more neural network models 110 on the dataset 302A. The set of materials 304A may be determined based on named entities (for example, reactants or products) that may be identified from the dataset 302A. The material information 304B may include information (for example, time-varying constraints related to the environment) associated with each material of the set of materials 304A.

The natural language model 112 may be a transformer-based model that may include an encoder 402A and a decoder 402B. The encoder 402A may generate the first set of embeddings 306A based on the set of materials 304A. For each material of the set of materials 304A, an embedding of the first set of embeddings 306A (represented as “C”) may be generated. The first set of embeddings 306A may indicate features of each material of the set of materials 304A. The encoder 402A may further generate the second set of embeddings 306B (represented as “C”) based on the material information 304B. Based on the information associated with each material of the set of materials 304A (included in the material information 304B), an embedding of the second set of embeddings 306B may be generated. The second set of embeddings 306B may indicate effect of each material of the set of materials 304A on resources of the living environment.

The generative AI model 114, i.e., each of the generator model 114A and the discriminator model 114B, may receive the first set of embeddings 306A and the second set of embeddings 306B as inputs. The generative AI model 114 may be trained based on the inputs. The generator model 114A may additionally receive a random input 404 (represented as “Z”) that may be generated by use of a set of Gaussian noise models. For a queried material (for example, a material of the set of materials 304A or any other material), the generator model 114A may generate an output (represented by “G (Z, C, C′)”). The discriminator model 114B may additionally receive the material information 304B and the generated output (i.e., “G (Z, C, C′)”) as inputs. Based on an application of the discriminator model 114B on the inputs, a result (represented by “R”) may be generated. The processor 204 may determine a labeler loss 406A (for example, generator loss) based on result and the output. Additionally, the processor 204 may determine an anti-labeler loss 406B. The generator model 114A may be further trained based on the labeler loss 406A. Based on the training, the output (i.e., “G (Z, C, C′)”) generated by the generator model 114A may be updated. The discriminator model 114B may generate a prediction (represented by “P”) which may indicate whether the queried material is sustainable or hazardous. The discriminator model 114B may further indicate a rationale (represented as “R”) behind the prediction. The prediction and the rationale behind the prediction may constitute sustainability information associated with the queried material.

It should be noted that the exemplary architecture 400 of FIG. 4 is for exemplary purposes and should not be construed to limit the scope of the disclosure.

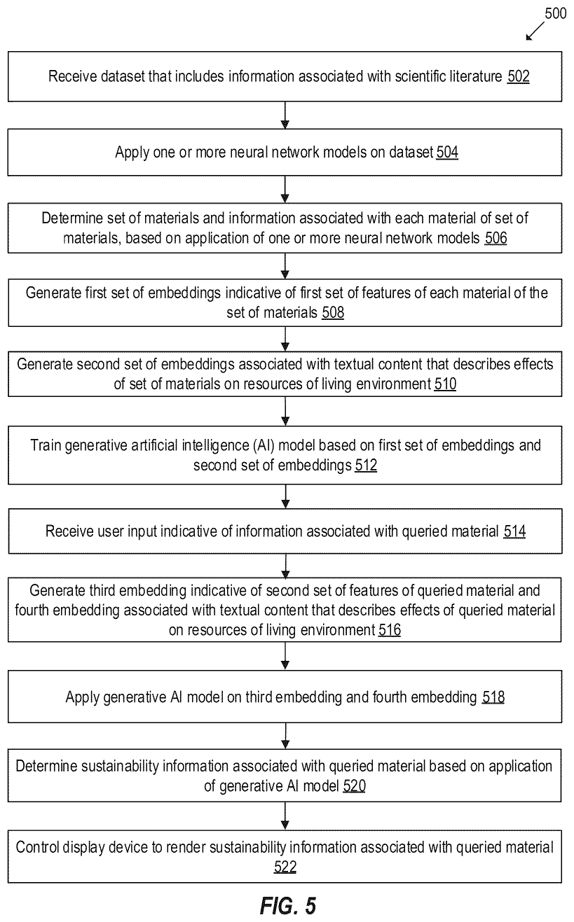

FIG. 5 is a diagram that illustrates a flowchart of an example method for AI-based sustainable material design, in accordance with an embodiment of the disclosure. FIG. 5 is described in conjunction with elements from FIG. 1, FIG. 2, FIG. 3, and FIG. 4. With reference to FIG. 5, there is shown a flowchart 500. The method illustrated in the flowchart 500 may start at 502 and may be performed by any suitable system, apparatus, or device, such as, by the example electronic device 102 of FIG. 1, or the processor 204 of FIG. 2. Although illustrated with discrete blocks, the steps and operations associated with at least one block of the flowchart 500 may be divided into additional blocks, combined into fewer blocks, or eliminated, depending on the particular implementation.

At block 502, a dataset, that includes information associated with scientific literature, may be received. In an embodiment, the processor 204 may be configured to receive the dataset that includes information associated with the scientific literature. Details of reception of the dataset including information associated with the scientific literature are further provided, for example, in FIG. 1 and FIG. 3.

At block 504, one or more neural network models 110 may be applied on the dataset. In an embodiment, the processor 204 may be configured to apply the one or more neural network models 110 on the dataset. Details of application of the one or more neural network models 110 on the received dataset are further provided, for example, in FIG. 1 and FIG. 3.

At block 506, a set of materials and information associated with each material of the set of materials may be determined based on the application of the one or more neural network models 110. In an embodiment, the processor 204 may be configured to determine the set of materials and information associated with each material of the set of materials, based on the application of the one or more neural network models 110. Details of determination of the set of materials and information associated with each material of the set of materials are further provided, for example, in FIG. 1 and FIG. 3.

At block 508, a first set of embeddings indicative of a first set of features of each material of the set of materials may be generated. In an embodiment, the processor 204 may be configured to generate the first set of embeddings indicative of the first set of features of each material of the set of materials. Details of generation of the first set of embeddings are further provided, for example, in FIG. 1, FIG. 3, and FIG. 4.

At block 510, a second set of embeddings associated with textual content that describes effects of the set of materials on resources of a living environment may be generated for the set of materials. In an embodiment, the processor 204 may be configured to generate, for the set of materials, the second set of embeddings indicative of the impact of the set of materials on the environment. Details of generation of the second set of embeddings are further provided, for example, in FIG. 1, FIG. 3, and FIG. 4.

At block 512, the generative AI model 114 may be trained based on the first set of embeddings and the second set of embeddings. In an embodiment, the processor 204 may be configured to train the generative AI model 114 based on the first set of embeddings and the second set of embeddings. Details of training of generative AI model 114 are further provided, for example, in FIG. 1 and FIG. 3.

At block 514, a user input, indicative of information associated with a queried material, may be received. In an embodiment, the processor 204 may be configured to receive the user input indicative of the information associated with the queried material. Details of reception of the user input indicative of the information associated with the queried material are further provided, for example, in FIG. 1, FIG. 3, and FIG. 4.

At block 516, a third embedding indicative of a second set of features of the queried material and a fourth embedding associated with textual content, that describes effects of the queried material on the resources of the living environment, may be generated. In an embodiment, the processor 204 may be configured to generate the third embedding indicative of the second set of features of the queried material and the fourth embedding associated with textual content, that describes effects of the queried material on the resources of the living environment. Details of generation of the third embedding and the fourth embedding are further provided, for example, in FIG. 1, FIG. 3, and FIG. 4.

At block 518, the generative AI model 114 may be applied on the third embedding and the fourth embedding. In an embodiment, the processor 204 may be configured to apply the generative AI model 114 on the third embedding and the fourth embedding. Details of application of the generative AI model 114 on the third embedding and the fourth embedding are further provided, for example, in FIG. 1, FIG. 3, and FIG. 4.

At block 520, sustainability information associated with the queried material may be determined based on the application of the generative AI model 114. In an embodiment, the processor 204 may be configured to determine the sustainability information associated with the queried material based on application of the generative AI model 114. Details of determination of the sustainability information associated with the queried material are further provided, for example, in FIG. 1, FIG. 3, and FIG. 4.

At block 522, a display device (such as the display device 210A) may be controlled to render the sustainability information associated with the queried material. In an embodiment, the processor 204 may be configured to control the display device 210A to render the sustainability information associated with the queried material. Details of controlling of the display device for rendering of the sustainability information is further provided, for example, in FIG. 1.

Although the flowchart 500 is illustrated as discrete operations, such as 502 504, 506, 508, 510, 512, 514, 516, 518, 520, and 522, the disclosure is not so limited. However, in certain embodiments, such discrete operations may be further divided into additional operations, combined into fewer operations, or eliminated, depending on the particular implementation without detracting from the essence of the disclosed embodiments.

Various embodiments of the disclosure may provide one or more non-transitory computer-readable storage media configured to store instructions that, in response to being executed, cause a system (such as the example electronic device 102) to perform operations. The operations may include receiving a receiving a dataset that may include information associated with scientific literature. The operations may further include applying one or more neural network models (such as the one or more neural network models 110) on the dataset. The operations may further include determining a set of materials (such as the set of materials 116) and information associated with each material of the set of materials, 116 based on the application of the one or more neural network models 110. The operations may further include generating a first set of embeddings (such as the first set of embeddings 118) indicative of a first set of features of each material of the set of materials 116. The operations may further include generating a second set of embeddings (such as the second set of embeddings 120) associated with textual content that describes effects of the set of materials 116 on resources of a living environment. The operations may further include training a generative AI model (such as the generative AI model 114) based on the first set of embeddings 118 and the second set of embeddings 120. The operations may further include receiving a user input indicative of information associated with a queried material (such as the queried material 122). The operations may further include generating a third embedding indicative of a second set of features of the queried material 122 and a fourth embedding associated with textual content that describes effects of the queried material 122 on the resources of the living environment. The operations may further include applying the trained generative AI model 114 on the third embedding and the fourth embedding. The operations may further include determining sustainability information associated with the queried material 122 based on the application of the generative AI model 114. The operations may further include controlling a display device (i.e., the display device 210) to render the sustainability information associated with the queried material 122.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, etc.) of the computing system. In some embodiments, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated. In this description, a “computing entity” may be any computing system as previously defined in the present disclosure, or any module or combination of modulates running on a computing system.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

All examples and conditional language recited in the present disclosure are intended for pedagogical objects to aid the reader in understanding the present disclosure and the concepts contributed by the inventor to furthering the art and are to be construed as being without limitation to such specifically recited examples and conditions. Although embodiments of the present disclosure have been described in detail, various changes, substitutions, and alterations could be made hereto without departing from the spirit and scope of the present disclosure.

What is claimed is:

1. A method, executed by a processor, comprising:
receiving a dataset that includes information associated with scientific literature;
applying one or more neural network models on the dataset;
determining a set of materials and information associated with each material of the set of materials, based on the application of the one or more neural network models;
generating a first set of embeddings indicative of a first set of features of each material of the set of materials;
generating a second set of embeddings associated with textual content that describes effects of the set of materials on resources of a living environment;
training a generative artificial intelligence (AI) model based on the first set of embeddings and the second set of embeddings;
receiving a user input indicative of information associated with a queried material;
generating a third embedding indicative of a second set of features of the queried material and a fourth embedding associated with textual content that describes effects of the queried material on the resources of the living environment;
applying the generative AI model on the third embedding and the fourth embedding;
determining sustainability information associated with the queried material based on the application of the generative AI model; and
controlling a display device to render the sustainability information associated with the queried material.

2. The method according to claim 1, further comprising:
applying a first neural network model of the one or more neural network models on the dataset;
identifying a set of reactants based on the application of the first neural network model;
applying a second neural network model of the one or more neural network models on the set of reactants; and
selecting a subset of reactants from the set of reactants based on the application of the second neural network model, wherein
the determination of the set of materials is further based on the identification of the set of reactants and the selection of the subset of reactants.

3. The method according to claim 2, further comprising:
extracting information associated with each reactant of the set of reactants based on the application of the first neural network model, wherein
the information associated with each material of the set of materials includes information associated with each reactant of one or more reactants of the set of reactants included in the corresponding material.

4. The method according to claim 3, wherein the determined information associated with each reactant of the set of reactants includes at least one of:
an organic structure of a corresponding reactant,
a decay rate associated with the corresponding reactant,
a biodegradability associated with the corresponding reactant,
one or more catalysts facilitating a chemical reaction that involves the corresponding reactant,
one or more products generated due to the chemical reaction,
a temperature requirement for triggering the chemical reaction, or
one or more precursors involved in the chemical reaction.

5. The method according to claim 1, further comprising:
applying a natural language model on each material of the set of materials and the information associated with each material of the set of materials, wherein
the generation of the first set of embeddings and the generation of the second set of embeddings are further based on application of the natural language model.

6. The method according to claim 1, wherein the generative AI model corresponds to a conditional generative adversarial network (GAN) model that includes a generator model and a discriminator model.

7. The method according to claim 6, wherein the generator model is trained to generate an output for each material of the set of materials such that the discriminator model classifies each material of the set of materials as sustainable.

8. The method according to claim 7, wherein the output is generated based on at least one of: a random input, the first set of embeddings, the second set of embeddings, a generator loss received from the discriminator model based on previously generated outputs, or the information associated with each material of the set of materials.

9. The method according to claim 7, wherein the discriminator model is configured to classify the material as sustainable or hazardous.

10. The method according to claim 9, wherein the classification of the discriminator model is based on at least one of: the output generated for each material of the set of materials, the first set of embeddings, the second set of embeddings, a discriminator loss generated based on previous classification results, and the information associated with each material of the set of materials.

11. The method according to claim 6, further comprising:
receiving, by the generator model, a first set of inputs including a random input, the third embedding, and the fourth embedding;
generating, by the generator model, based on the first set of inputs, an output;
receiving, by the discriminator model, a second set of inputs including the output, the third embedding, and the fourth embedding; and
classifying, by the discriminator model, the queried material as sustainable or hazardous based on the second set of inputs.

12. The method according to claim 1, wherein the determined sustainability information associated with the queried material corresponds to a first indication that specifies whether the queried material is sustainable or hazardous and a second indication that explains a rationale behind the first indication.

13. One or more non-transitory computer-readable storage media configured to store instructions that, in response to being executed, cause an electronic device to perform operations, the operations comprising:
receiving a dataset that includes information associated with scientific literature;
applying one or more neural network models on the dataset;
determining a set of materials and information associated with each material of the set of materials, based on the application of the one or more neural network models;
generating a first set of embeddings indicative of a first set of features of each material of the set of materials;
generating a second set of embeddings associated with textual content that describes effects the set of materials on resources of a living environment;
training a generative artificial intelligence (AI) model based on the first set of embeddings and the second set of embeddings;
receiving a user input indicative of information associated with a queried material;
generating a third embedding indicative of a second set of features of the queried material and a fourth embedding associated with textual content that describes effects of the queried material on the resources of the living environment;
applying the generative AI model on the third embedding and the fourth embedding;
determining sustainability information associated with the queried material based on the application of the generative AI model; and
controlling a display device to render the sustainability information associated with the queried material.

14. The non-transitory computer-readable storage medium according to claim 13, wherein the operations comprise:
applying a first neural network model of the one or more neural network models on the dataset;
identifying a set of reactants based on the application of the first neural network model;
applying a second neural network model of the one or more neural network models on the set of reactants; and
selecting a subset of reactants from the set of reactants based on the application of the second neural network model, wherein
the determination of the set of materials is further based on the identification of the set of reactants and the selection of the subset of reactants.

15. The non-transitory computer-readable storage medium according to claim 14, wherein the operations comprise:
extracting information associated with each reactant of the set of reactants based on the application of the first neural network model, wherein
the information associated with each material of the set of materials includes information associated with each reactant of one or more reactants of the set of reactants included in the corresponding material.

16. The non-transitory computer-readable storage medium according to claim 15, wherein the determined information associated with each reactant of the set of reactants includes at least one of:
an organic structure of a corresponding reactant,
a decay rate associated with the corresponding reactant,
a biodegradability associated with the corresponding reactant,
one or more catalysts facilitating a chemical reaction that involves the corresponding reactant,
one or more products generated due to the chemical reaction,
a temperature requirement for triggering the chemical reaction, or
one or more precursors involved in the chemical reaction.

17. The non-transitory computer-readable storage medium according to claim 13, wherein the operations comprise:
applying a natural language model on each material of the set of materials and the information associated with each material of the set of materials, wherein
the determination of the first set of embeddings and the determination of the second set of embeddings are further based on application of the natural language model.

18. The non-transitory computer-readable storage medium according to claim 13, wherein the generative AI model corresponds to a conditional generative adversarial network (GAN) model that includes a generator model and a discriminator model.

19. The non-transitory computer-readable storage medium according to claim 13, wherein the determined sustainability information associated with the queried material corresponds to a first indication that specifies whether the queried material is sustainable or hazardous and a second indication that explains a rationale behind the first indication.

20. An electronic device, comprising:
a memory configured to store instructions; and
a processor, coupled to the memory, configured to execute the instructions to perform a process comprising:
receiving a dataset that includes information associated with scientific literature;
applying one or more neural network models on the dataset;
determining a set of materials and information associated with each material of the set of materials, based on the application of the one or more neural network models;
generating a first set of embeddings indicative of a first set of features of each material of the set of materials;
generating a second set of embeddings associated with textual content that describes effects of the set of materials on resources of a living environment;
training a generative artificial intelligence (AI) model based on the first set of embeddings and the second set of embeddings;
receiving a user input indicative of information associated with a queried material;
generating a third embedding indicative of a second set of features of the queried material and a fourth embedding associated with textual content that describes effects of the queried material on the resources of the living environment;
applying the generative AI model on the third embedding and the fourth embedding;
determining sustainability information associated with the queried material based on the application of the generative AI model; and
controlling a display device to render the sustainability information associated with the queried material.
